(19) United States
(12) Patent Application Publication
(10) Pub. No.: US 2018/0338995 A1
(43) Pub. Date: Nov. 29, 2018

(54) SYSTEMS AND METHODS FOR TREATMENTS USING HYDROGEN AND/OR NOBLE GASES

(71) Applicant: Perricone Hydrogen Water Company, LLC, Meriden, CT (US)

(72) Inventor: Nicholas V. Perricone, Madison, CT (US)

(73) Assignee: Perricone Hydrogen Water Company, LLC, Meriden, CT (US)

(21) Appl. No.: 15/986,885

(22) Filed: May 23, 2018

Related U.S. Application Data
(60) Provisional application No. 62/510,114, filed on May 23, 2017.

(51) Int. Cl. A61K 33/00; A61K 9/08; A61K 9/00; A61P 25/28

(52) U.S. Cl. CPC A61K 33/00; A61P 25/28; A61K 9/0053; A61K 9/08

(57) ABSTRACT

Embodiments described herein generally relate to systems and methods for treatments using xenon and hydrogen. In some embodiments, liquids comprising xenon and hydrogen are provided. Such liquids may be useful, for example, for the treatment of animal and human diseases, for improvement in athletic performance, for the enhancement of the overall health of a subject, or the like. The liquids (e.g., aqueous solutions) and articles described herein may be administered to a subject, e.g., drunk by a subject. Some embodiments relate to liquids disposed within a container (e.g., a sealed container), such that the liquid comprises hydrogen gas and a noble gas dissolved or otherwise contained (e.g., infused) in the liquid. In certain embodiments, the hydrogen gas and/or noble gas may be present in the liquid at a particular concentration (e.g., a physiologically relevant concentration). Advantageously, the compositions, articles, and methods described herein may provide liquids containing hydrogen gas and a noble gas that are shelf-stable (e.g., maintain a relatively stable concentration of hydrogen gas and/or noble gas(es)) for relatively long periods of time (e.g., at least 7 days).

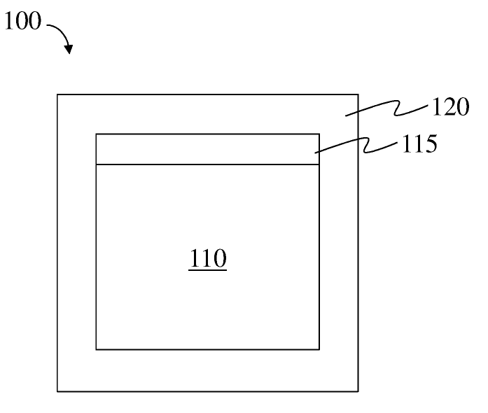

### RELATED APPLICATIONS

This application claims the benefit of U.S. Provisional Patent Application Ser. No. 62/510,114, filed May 23, 2017, entitled “Systems and Methods for Treatments Using Hydrogen and/or Noble Gases,” by Perricone, which is incorporated herein by reference in its entirety.

### FIELD OF THE INVENTION

The present invention generally relates to systems and methods for treatments using xenon and hydrogen.

### BACKGROUND

H₂ has been shown to have positive effects on animal and human physiology and disease states. H₂ can be administered to a subject in the form of, for example, a gas, an infusion, a topical solution, or through the drinking of H₂-enriched water. Production of hydrogen-rich water has been accomplished by several methods, ranging from large-scale, but less self-stable, manufacturing techniques to small-volume single use devices for locally generating hydrogen gas.

### SUMMARY OF THE INVENTION

The present invention provides systems and methods for treatments using xenon and hydrogen. The subject matter of the present invention involves, in some cases, interrelated products, alternative solutions to a particular problem, and/or a plurality of different uses of one or more systems and/or articles.

In one aspect, the present invention is directed to an article. According to one set of embodiments, the article includes an aqueous solution contained within a container. In some embodiments, the aqueous solution comprises dissolved hydrogen gas in a concentration greater than 1 ppm and dissolved xenon gas in a concentration greater than 1 ppm. In addition, in certain cases, the article may be configured for administering the aqueous solution to a subject.

In another aspect, the present invention is directed to a method. In one set of embodiments, the method comprises administering an aqueous solution to a subject. In some instances, the aqueous solution comprises dissolved hydrogen gas in a concentration greater than 1 ppm and dissolved xenon gas in a concentration greater than 1 ppm.

The method, in another set of embodiments, includes administering an aqueous solution to a subject. The aqueous solution may arise from a sealed container containing the aqueous solution and a headspace. In some cases, the aqueous solution comprises dissolved hydrogen gas and dissolved xenon gas, and in certain instances, hydrogen gas is present in the headspace in a concentration greater than or equal to 0.00001 vol %, 0.0001 vol %, 0.001 vol %, 0.01 vol %, 0.1 vol %, or 1 vol % versus the total volume of the headspace.

In yet another set of embodiments, the method includes an act of administering an aqueous solution to a subject. The aqueous solution may arise from a sealed container containing the aqueous solution and a headspace. In some cases, the aqueous solution comprises dissolved hydrogen gas and dissolved xenon gas, and in certain instances, xenon gas is present in the headspace in a concentration greater than or equal to 0.00001 vol %, 0.0001 vol %, 0.001 vol %, 0.01 vol %, 0.1 vol %, or 1 vol % versus the total volume of the headspace.

The method, in accordance with still another set of embodiments, includes administering an aqueous solution to a subject, where the aqueous solution comprises dissolved hydrogen gas in an amount of greater than 1 ppm and dissolved xenon gas in an amount of greater than 1 ppm, and wherein the aqueous solution is non-toxic.

In one set of embodiments, the method includes administering an aqueous solution to a subject, where the aqueous solution consists essentially of water, dissolved hydrogen gas in a concentration greater than 1 ppm, and a dissolved noble gas in a concentration greater than 1 ppm.

The method, in another set of embodiments, includes administering, to a subject proximate in time, an anesthetic and an aqueous solution comprising dissolved hydrogen gas and dissolved xenon gas.

Still another set of embodiments is generally directed to a method of administering, to a subject in need of a neuroprotectant, an aqueous solution comprising dissolved hydrogen gas and dissolved xenon gas.

In yet another set of embodiments, the method includes administering, to a subject having or at risk of hypoxia, an aqueous solution comprising dissolved hydrogen gas and dissolved xenon gas.

In still another set of embodiments, the method includes administering, to a subject having or at risk of hypothermia, an aqueous solution comprising dissolved hydrogen gas and dissolved xenon gas.

The method, in another set of embodiments, includes administering, to a subject having or at risk of ischemia, an aqueous solution comprising dissolved hydrogen gas and dissolved xenon gas.

According to yet another set of embodiments, the method includes administering, to a subject having or at risk of oxygen glucose deprivation, an aqueous solution comprising dissolved hydrogen gas and dissolved xenon gas.

In another set of embodiments, the method includes administering, to a subject having or at risk of neuropathic pain, an aqueous solution comprising dissolved hydrogen gas and dissolved xenon gas.

The method, in one set of embodiments, includes administering, to a subject having or at risk of a muscular disease, an aqueous solution comprising dissolved hydrogen gas and dissolved xenon gas.

The method, in accordance with another set of embodiments, includes administering, to a subject having or at risk of neurodegeneration, an aqueous solution comprising dissolved hydrogen gas and dissolved xenon gas.

In still another set of embodiments, the method includes administering, to a subject having or at risk of neuromuscular disease, an aqueous solution comprising dissolved hydrogen gas and dissolved xenon gas.

Yet another set of embodiments is generally directed to a method comprising administering, to a subject having or at risk of a metabolic condition, an aqueous solution comprising dissolved hydrogen gas and dissolved xenon gas.

The method, in another set of embodiments, includes administering, to a subject having or at risk of cardiovascular disease, an aqueous solution comprising dissolved hydrogen gas and dissolved xenon gas.

In another set of embodiments, the method comprises administering, to a subject having or at risk of an inflammatory disease, an aqueous solution comprising dissolved hydrogen gas and dissolved xenon gas.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF THE DRAWINGS

The FIGURE is a schematic drawing illustrating an article comprising a liquid disposed within a container, according to one set of embodiments.

Other aspects, embodiments and features of the invention will become apparent from the following detailed description when considered in conjunction with the accompanying drawings. The accompanying figures are schematic and are not intended to be drawn to scale. For purposes of clarity, not every component is labeled in every figure, nor is every component of each embodiment of the invention shown where illustration is not necessary to allow those of ordinary skill in the art to understand the invention. All patent applications and patents incorporated herein by reference are incorporated by reference in their entirety. In case of conflict, the present specification, including definitions, will control.

### DETAILED DESCRIPTION

Embodiments described herein generally relate to systems and methods for treatments using xenon and hydrogen. In some embodiments, liquids comprising xenon and hydrogen are provided. Such liquids may be useful, for example, for the treatment of animal and human diseases, for improvement in athletic performance, for the enhancement of the overall health of a subject, or the like. The liquids (e.g., aqueous solutions) and articles described herein may be administered to a subject, e.g., drunk by a subject. Some embodiments relate to liquids disposed within a container (e.g., a sealed container), such that the liquid comprises hydrogen gas and a noble gas dissolved or otherwise contained (e.g., infused) in the liquid. In certain embodiments, the hydrogen gas and/or noble gas may be present in the liquid at a particular concentration (e.g., a physiologically relevant concentration). Advantageously, the compositions, articles, and methods described herein may provide liquids containing hydrogen gas and a noble gas that are shelf-stable (e.g., maintain a relatively stable concentration of hydrogen gas and/or noble gas(es)) for relatively long periods of time (e.g., at least 7 days).

In one aspect, generally, the composition comprises a liquid comprising hydrogen gas and a noble gas. In some embodiments, the liquid comprises hydrogen gas and a noble gas dissolved in water. The liquid may be water, optionally with a variety of additives, such as sugar, electrolytes, caffeine, salt(s), flavoring, vitamins, herbs, amino acids, tea extracts, seed extracts, fruit extracts. The liquid may be any of a variety of drinkable liquids, such as a fruit juice, coffee, tea, a sports drink, an energy drink, soda pop, milk, an alcoholic drink, or the like. In certain embodiments, the liquid may be configured to be administered (e.g., orally, intravenously, etc.) to a subject (e.g., in a clinical setting). For example, the liquid may be in the form of an intravenous fluid (e.g., saline).

The hydrogen gas may be present in the liquid in a particular amount (e.g., an amount that would have a significant effect on the body of a subject). The hydrogen gas may be dissolved and/or suspended in the liquid. In some embodiments, the hydrogen gas is present in the liquid in an amount of greater than or equal to 0.1 ppm, greater than or equal to 0.2 ppm, greater than or equal to 0.3 ppm, greater than or equal to 0.4 ppm, greater than or equal to 0.5 ppm, greater than or equal to 0.6 ppm, greater than or equal to 0.7 ppm, greater than or equal to 0.8 ppm, greater than or equal to 0.9 ppm, greater than or equal to 1 ppm, greater than or equal to 1.1 ppm, greater than or equal to 1.2 ppm, greater than or equal to 1.25 ppm, greater than or equal to 1.3 ppm, greater than or equal to 1.4 ppm, greater than or equal to 1.5 ppm, greater than or equal to 1.6 ppm, greater than or equal to 1.7 ppm, greater than or equal to 1.75 ppm, greater than or equal to 1.8 ppm, greater than or equal to 1.9 ppm, greater than or equal to 2 ppm, greater than or equal to 2.25 ppm, greater than or equal to 2.5 ppm, greater than or equal to 2.75 ppm, greater than or equal to 3 ppm, greater than or equal to 3.25 ppm, greater than or equal to 3.5 ppm, greater than or equal to 3.75 ppm, greater than or equal to 4 ppm, greater than or equal to 4.25 ppm, greater than or equal to 4.5 ppm, or greater than or equal to 4.75 ppm.

In certain embodiments, the hydrogen gas is present in the liquid in an amount of less than or equal to 5 ppm, less than or equal to 4.75 ppm, less than or equal to 4.5 ppm, less than or equal to 4.25 ppm, less than or equal to 4 ppm, less than or equal to 3.75 ppm, less than or equal to 3.5 ppm, less than or equal to 3.25 ppm, less than or equal to 3 ppm, less than or equal to 2.75 ppm, less than or equal to 2.5 ppm, less than or equal to 2.25 ppm, less than or equal to 2 ppm, less than or equal to 1.9 ppm, less than or equal to 1.8 ppm, less than or equal to 1.75 ppm, less than or equal to 1.7 ppm, less than or equal to 1.6 ppm, less than or equal to 1.5 ppm, less than or equal to 1.4 ppm, less than or equal to 1.3 ppm, less than or equal to 1.25 ppm, less than or equal to 1.2 ppm, less than or equal to 1.1 ppm, less than or equal to 1 ppm, less than or equal to 0.9 ppm, less than or equal to 0.8 ppm, less than or equal to 0.7 ppm, less than or equal to 0.6 ppm, less than or equal to 0.5 ppm, less than or equal to 0.4 ppm, less than or equal to 0.3 ppm, or less than or equal to 0.2 ppm. Combinations of the above-referenced ranges are also possible (e.g., greater than or equal to 0.1 ppm and less than or equal to 5 ppm, greater than or equal to 1.5 ppm and less than or equal to 2 ppm). Other ranges are also possible.

Non-limiting examples of suitable noble gases that may be present in the liquid include helium, neon, argon, krypton, xenon, and radon. In a particular set of embodiments, the noble gas is xenon gas. In some cases, two or more noble gases may be present, e.g., each independently at the concentrations below. The noble gas may be substantially dissolved and/or suspended in the liquid. For example, the mole fraction solubility of xenon in water at 25° C. and 1 atm is generally 7.890×10⁻⁵. In some embodiments, the amount of noble gas dissolved in the liquid is greater than the amount of noble gas that would be dissolved in the liquid at the mole fraction solubility of the noble gas in water determined at 25° C. and 1 atm. For example, the liquid may be under a pressure greater than 1 atm and/or a temperature greater than 25° C., e.g., as discussed below, which may facilitate greater amounts.

The noble gas may be present in the liquid in a particular amount. For example, in some embodiments, the noble gas is present in an amount of greater than or equal to 1 ppm, greater than or equal to 1.25 ppm, greater than or equal to 1.5 ppm, greater than or equal to 1.75 ppm, greater than or equal to 2 ppm, greater than or equal to 2.25 ppm, greater than or equal to 2.5 ppm, greater than or equal to 2.75 ppm, greater than or equal to 3 ppm, greater than or equal to 3.25 ppm, greater than or equal to 3.5 ppm, greater than or equal to 3.75 ppm, greater than or equal to 4 ppm, greater than or equal to 4.25 ppm, greater than or equal to 4.5 ppm, greater than or equal to 4.75 ppm, greater than or equal to 5 ppm, greater than or equal to 5.5 ppm, greater than or equal to 6 ppm, greater than or equal to 6.5 ppm, greater than or equal to 7 ppm, greater than or equal to 7.5 ppm, greater than or equal to 8 ppm, greater than or equal to 8.5 ppm, greater than or equal to 9 ppm, greater than or equal to 9.5 ppm, greater than or equal to 10 ppm, greater than or equal to 11 ppm, greater than or equal to 12 ppm, greater than or equal to 13 ppm, greater than or equal to 14 ppm, greater than or equal to 15 ppm, greater than or equal to 16 ppm, greater than or equal to 17 ppm, greater than or equal to 18 ppm, or greater than or equal to 19 ppm.

In certain embodiments, the noble gas is present in the liquid in an amount of less than or equal to 20 ppm, less than or equal to 19 ppm, less than or equal to 18 ppm, less than or equal to 17 ppm, less than or equal to 16 ppm, less than or equal to 15 ppm, less than or equal to 14 ppm, less than or equal to 13 ppm, less than or equal to 12 ppm, less than or equal to 11 ppm, less than or equal to 10 ppm, less than or equal to 9.5 ppm, less than or equal to 9 ppm, less than or equal to 8.5 ppm, less than or equal to 8 ppm, less than or equal to 7.5 ppm, less than or equal to 7 ppm, less than or equal to 6.5 ppm, less than or equal to 5 ppm, less than or equal to 4.75 ppm, less than or equal to 4.5 ppm, less than or equal to 4.25 ppm, less than or equal to 4 ppm, less than or equal to 3.75 ppm, less than or equal to 3.5 ppm, less than or equal to 3.25 ppm, less than or equal to 3 ppm, less than or equal to 2.75 ppm, less than or equal to 2.5 ppm, less than or equal to 2.25 ppm, less than or equal to 2 ppm, less than or equal to 1.75 ppm, less than or equal to 1.5 ppm, or less than or equal to 1.25 ppm. Combinations of the above-referenced ranges are also possible (e.g., greater than or equal to 1 ppm and less than or equal to 20 ppm, greater than or equal to 10 ppm and less than or equal to 15 ppm). Other ranges are also possible. The noble gas may include xenon, and/or other gases as discussed herein.

In some cases, the liquid may be present in a container. For example, as illustrated schematically in the FIGURE, article 100 may comprise a liquid (for example, an aqueous solution) 110 disposed in container 120. In certain embodiments, the container may be sealed, e.g., to the external atmosphere. For example, in certain embodiments, the container may be sealed such that the liquid and/or gases (e.g., hydrogen gas, and/or xenon gas) within the container are not able to substantially exit the container. In some cases, the seal may be removable (e.g., such that the liquid may be removed from the container and/or orally administered to a subject, e.g., drunk). For example, in an exemplary embodiment, the container is a can and the can may be unsealed by breaking the seal of the can (e.g., via a pull-tab, push-tab, or stay-tab associated with the seal). In another exemplary embodiment, the container is a bottle or pouch, and the container may be unsealed by removing a cap associated with an opening of the container. Upon unsealing of the container, the liquid may be ingested (e.g., drunk) by the subject. In some cases, the container may be used for intravenous infusion, or other administration techniques such as those described herein.

Non-limiting examples of suitable types of containers include cans (e.g., aluminum or tin cans), bottles, jars, pouches, boxes, bags, and capsules (e.g., a liquid gel capsule). Other containers are also possible and those of ordinary skill in the art would be capable of selecting suitable containers based upon the teachings of this specification. The container may also comprise any suitable material. For example, in some embodiments, the container may comprise a material such as metal (e.g., aluminum, tin, iron), metal alloys (e.g., steel), polymer (e.g., polyethylene, polystyrene, polypropylene, polyether ether ketones, polyethylene terephthalate, polyvinylchloride), glass (e.g., borosilicate glass), resin, and combinations thereof. In certain embodiments, the fluid is present in the container is at or near atmospheric pressure. In some cases, however, the container is able to contain an elevated pressure therein (e.g., a pressure greater than atmospheric pressure).

In addition, in some embodiments, one or more coatings or other materials may be used to facilitate retention of gases within the container, e.g., such materials may be relatively gas-impermeable. A variety of gas-impermeable materials may be readily obtained commercially, and coated onto a surface of the container and/or embedded within the materials forming the container. Non-limiting examples of gas-impermeable materials include polyester, nylon (e.g., MXD6 nylon or nylon 6), ethylene vinyl alcohol (EVA), silicon oxides (SiOₓ), or the like. Thus, in some embodiments, the liquid is contained within a container at a particular pressure that may be greater than atmospheric pressure. The pressure may be created within the container using any of a variety of gases, including air, nitrogen, carbon dioxide, water vapor, hydrogen gas, one or more noble gases (such as xenon), or the like, as well as combinations of these and/or other suitable gases. Such gases may be at equilibrium with the liquid within the container. In addition, in some cases, one or more of the gases may be present in an amount such that at equilibrium, those gases are dissolved within the liquid at saturation concentrations.

For example, in certain embodiments, the container contains a pressure at least 1 psi (1 psi is about 6894.757 Pa), at least 2 psi, at least 3 psi, at least 5 psi, at least 7 psi, at least 10 psi, at least 12 psi, at least 15 psi, at least 18 psi, at least 20 psi, at least 25 psi, at least 30 psi, at least 35 psi, at least 40 psi, or at least 45 psi greater than atmospheric pressure. In some embodiments, the container contains a pressure of less than or equal to 50 psi, less than or equal to 45 psi, less than or equal to 40 psi, less than or equal to 35 psi, less than or equal to 30 psi, less than or equal to 25 psi, less than or equal to 20 psi, less than or equal to 18 psi, less than or equal to 15 psi, less than or equal to 12 psi, less than or equal to 10 psi, less than or equal to 7 psi, less than or equal to 5 psi, less than or equal to 3 psi, or less than or equal to 2 psi greater than atmospheric pressure. Combinations of the above-referenced ranges are also possible (e.g., at least 1 psi and less than or equal to 50 psi greater than atmospheric pressure). Other ranges are also possible.

In some embodiments, the article comprises a gaseous headspace (e.g., a gaseous headspace present within the container). For example, referring again to the FIGURE, in some cases, article 100 comprises gaseous headspace 115. The article may comprise any suitable amount of headspace within the container. In some embodiments, the headspace occupies greater than or equal to 0.1 vol %, greater than or equal to 0.2 vol %, greater than or equal to 0.25 vol %, greater than or equal to 0.5 vol %, greater than or equal to 0.75 vol %, greater than or equal to 1 vol %, greater than or equal to 1.25 vol %, greater than or equal to 1.5 vol %, greater than or equal to 1.75 vol %, greater than or equal to 2 vol %, greater than or equal to 2.25 vol %, greater than or equal to 2.5 vol %, greater than or equal to 3 vol %, greater than or equal to 3.5 vol %, greater than or equal to 4 vol %, or greater than or equal to 4.5 vol % of the volume contained by the container. In certain embodiments, the headspace occupies less than or equal to 5 vol %, less than or equal to 4.5 vol %, less than or equal to 4 vol %, less than or equal to 3.5 vol %, less than or equal to 3 vol %, less than or equal to 2.5 vol %, less than or equal to 2.25 vol %, less than or equal to 2 vol %, less than or equal to 1.75 vol %, less than or equal to 1.5 vol %, less than or equal to 1.25 vol %, less than or equal to 1 vol %, less than or equal to 0.75 vol %, less than or equal to 0.5 vol %, less than or equal to 0.25 vol %, or less than or equal to 0.2 vol % of the volume contained by the container. Combinations of the above-referenced ranges are also possible (e.g., greater than or equal to 0.1 vol % and less than or equal to 5 vol %). Other ranges are also possible.

In some embodiments, the gaseous headspace comprises hydrogen gas. In certain embodiments, the gaseous headspace comprises a noble gas such as xenon gas. In some cases, both hydrogen gas and the noble gas (e.g., xenon gas) may be present in the headspace. As mentioned, other gases (including other noble gases) may be present as well, e.g., as discussed herein. In some embodiments, when hydrogen gas and/or a noble gas such as xenon gas are present in the headspace, the concentration of hydrogen gas and/or xenon gas greater than the concentration of the gas that would result from the saturation vapor pressure of the gas.

For example, in some embodiments, the headspace comprises greater than or equal to 0.00001 vol %, greater than or equal to 0.00005 vol %, greater than or equal to 0.0001 vol %, greater than or equal to 0.0005 vol %, greater than or equal to 0.001 vol %, greater than or equal to 0.005 vol %, greater than or equal to 0.01 vol %, greater than or equal to 0.05 vol %, greater than or equal to 0.1 vol %, greater than or equal to 0.5 vol %, greater than or equal to 1 vol %, greater than or equal to 2 vol %, greater than or equal to 5 vol %, greater than or equal to 10 vol %, greater than or equal to 20 vol %, greater than or equal to 30 vol %, greater than or equal to 40 vol %, greater than or equal to 50 vol %, or greater than or equal to 60 vol % xenon gas (and/or other noble gases) versus the total volume of the headspace. In certain embodiments, xenon gas (and/or other noble gases) is present in the headspace in an amount less than or equal to 70 vol %, less than or equal to 60 vol %, less than or equal to 50 vol %, less than or equal to 40 vol %, less than or equal to 30 vol %, less than or equal to 20 vol %, less than or equal to 10 vol %, less than or equal to 5 vol %, less than or equal to 2 vol %, less than or equal to 1 vol %, less than or equal to 0.5 vol %, less than or equal to 0.1 vol %, less than or equal to 0.05 vol %, less than or equal to 0.01 vol %, less than or equal to 0.005 vol %, less than or equal to 0.001 vol %, less than or equal to 0.0005 vol %, less than or equal to 0.0001 vol %, or less than or equal to 0.00005 vol % versus the total volume of the headspace. Combinations of the above-referenced ranges are also possible (e.g., greater than or equal to 0.00001 vol % and less than or equal to 10 vol %). Other ranges are also possible.

In certain embodiments, the headspace comprises greater than or equal to 0.00001 vol %, greater than or equal to 0.00005 vol %, greater than or equal to 0.0001 vol %, greater than or equal to 0.0005 vol %, greater than or equal to 0.001 vol %, greater than or equal to 0.005 vol %, greater than or equal to 0.01 vol %, greater than or equal to 0.05 vol %, greater than or equal to 0.1 vol %, greater than or equal to 0.5 vol %, greater than or equal to 1 vol %, greater than or equal to 2 vol %, or greater than or equal to 5 vol % hydrogen gas versus the total volume of the headspace. In certain embodiments, hydrogen gas is present in the headspace in an amount less than or equal to 10 vol %, less than or equal to 5 vol %, less than or equal to 2 vol %, less than or equal to 1 vol %, less than or equal to 0.5 vol %, less than or equal to 0.1 vol %, less than or equal to 0.05 vol %, less than or equal to 0.01 vol %, less than or equal to 0.005 vol %, less than or equal to 0.001 vol %, less than or equal to 0.0005 vol %, less than or equal to 0.0001 vol %, or less than or equal to 0.00005 vol % versus the total volume of the headspace. Combinations of the above-referenced ranges are also possible (e.g., greater than or equal to 0.00001 vol % and less than or equal to 10 vol %). Other ranges are also possible.

In one set of embodiments, the liquid within the sealed container fills greater than or equal to 50 vol %, greater than or equal to 75 vol %, greater than or equal to 80 vol %, greater than or equal to 85 vol %, greater than or equal to 90 vol %, greater than or equal to 92 vol %, greater than or equal to 95 vol %, greater than or equal to 98 vol %, greater than or equal to 99 vol %, greater than or equal to 99.5 vol %, or greater than or equal to 99.9 vol % of the volume of the sealed container. In some cases, the volume of the liquid may be less than or equal to 99.99 vol %, less than or equal to 99.9 vol %, less than or equal to 99.5 vol %, less than or equal to 99 vol %, less than or equal to 98 vol %, less than or equal to 95 vol %, less than or equal to 92 vol %, less than or equal to 90 vol %, less than or equal to 85 vol %, less than or equal to 80 vol %, or less than or equal to 75 vol % of the volume of the sealed container. Combinations of the above-referenced ranges are also possible (e.g., greater than or equal to 50 vol % and less than or equal to 99.99 vol %).

In some embodiments, the articles (e.g., a sealed container) described herein are configured to have a relatively long shelf life with respect to the gases contained therein. In certain embodiments, the hydrogen gas and noble gas (e.g., xenon gas) does not substantially leak from the sealed container for at least 7 days, or longer (e.g., 14 days, 28 days, 56 days, etc.).

For example, in some embodiments, greater than or equal to 50 vol %, greater than or equal to 75 vol %, greater than or equal to 80 vol %, greater than or equal to 85 vol %, greater than or equal to 90 vol %, greater than or equal to 92 vol %, greater than or equal to 95 vol %, greater than or equal to 98 vol %, greater than or equal to 99 vol %, greater than or equal to 99.5 vol %, or greater than or equal to 99.9 vol % of the hydrogen gas and/or xenon gas (and/or other noble gases) is present in the sealed container and/or in the headspace 7 days after sealing of the container (including the liquid comprising the hydrogen gas and the noble gas). In certain embodiments, less than or equal to 99.99 vol %, less than or equal to 99.9 vol %, less than or equal to 99.5 vol %, less than or equal to 99 vol %, less than or equal to 98 vol %, less than or equal to 95 vol %, less than or equal to 92 vol %, less than or equal to 90 vol %, less than or equal to 85 vol %, less than or equal to 80 vol %, or less than or equal to 75 vol % of the hydrogen gas and/or xenon gas (and/or other noble gases) is present in the sealed container and/or in the headspace 7 days after sealing of the container (including within the liquid comprising the hydrogen gas and the noble gas). Combinations of the above-referenced ranges are also possible (e.g., greater than or equal to 50 vol % and less than or equal to 99.99 vol %). Other ranges are also possible. In some embodiments, one or more additives may be present. Non-limiting examples of additives include sugar, electrolytes, caffeine, salt(s), flavoring, vitamins, herbs, amino acids, tea extracts, seed extracts, fruit extracts, and combinations thereof. The one or more additives may be present in any suitable amount. For example, in some embodiments, the additive is present in the liquid in an amount of greater than or equal to 0.1 vol %, greater than or equal to 0.2 vol %, greater than or equal to 0.25 vol %, greater than or equal to 0.5 vol %, greater than or equal to 0.75 vol %, greater than or equal to 1 vol %, greater than or equal to 1.25 vol %, greater than or equal to 1.5 vol %, greater than or equal to 1.75 vol %, greater than or equal to 2 vol %, greater than or equal to 2.25 vol %, greater than or equal to 2.5 vol %, greater than or equal to 3 vol %, greater than or equal to 3.5 vol %, greater than or equal to 4 vol %, or greater than or equal to 4.5 vol % versus the total volume of the liquid.

In certain embodiments, the additive is present in the liquid solution in an amount less than or equal to 5 vol %, less than or equal to 4.5 vol %, less than or equal to 4 vol %, less than or equal to 3.5 vol %, less than or equal to 3 vol %, less than or equal to 2.5 vol %, less than or equal to 2.25 vol %, less than or equal to 2 vol %, less than or equal to 1.75 vol %, less than or equal to 1.5 vol %, less than or equal to 1.25 vol %, less than or equal to 1 vol %, less than or equal to 0.75 vol %, less than or equal to 0.5 vol %, less than or equal to 0.25 vol %, or less than or equal to 0.2 vol % versus the total volume of the liquid. Combinations of the above-referenced ranges are also possible (e.g., greater than or equal to 0.1 vol % and less than or equal to 5 vol %). Other ranges are also possible.

As mentioned, the liquid may be any of a variety of drinkable liquids in various embodiments, such as water, a fruit juice, coffee, tea, a sports drink, an energy drink, soda pop, milk, an alcoholic drink, etc. In some cases, the liquid may be in the form of administrable to a subject (e.g., in an intravenous bag or pouch for intravenous delivery such as comprising saline).

In an exemplary embodiment, the article (e.g., the container) is configured for intravenous delivery of the liquid contained therein to a subject and comprises saline (e.g., NaCl dissolved in water). In some such embodiments, the additive is NaCl. In some cases, the liquid may be normal saline (i.e. 0.9 wt %/vol % NaCl per total volume of the water present in the liquid) and comprises hydrogen gas and xenon gas. In some embodiments, NaCl is present in the liquid in an amount of greater than or equal to 0.1 wt %, greater than or equal to 0.2 wt %, greater than or equal to 0.3 wt %, greater than or equal to 0.5 wt %, greater than or equal to 0.7 wt %, greater than or equal to 0.9 wt %, greater than or equal to 1 wt %, greater than or equal to 1.2 wt %, greater than or equal to 1.5 wt %, greater than or equal to 1.7 wt %, or greater than or equal to 2 wt % per total volume of the water present in the liquid). In certain embodiments, NaCl is present in the liquid in an amount of less than or equal to 2.5 wt %, less than or equal to 2 wt %, less than or equal to 1.7 wt %, less than or equal to 1.5 wt %, less than or equal to 1.2 wt %, less than or equal to 1 wt %, less than or equal to 0.9 wt %, less than or equal to 0.7 wt %, less than or equal to 0.5 wt %, less than or equal to 0.3 wt %, or less than or equal to 0.2 wt %. Combinations of the above-referenced ranges are also possible (e.g., greater than or equal to 0.1 wt % and less than or equal to 2.5 wt %, greater than or equal to 0.7 wt % and less than or equal to 1 wt %). Other ranges are also possible.

In some embodiments, the liquid (e.g., the aqueous solution) comprises hydrogen gas, a noble gas (e.g., xenon gas), and one or more additives, in the ranges listed above with the remainder of the liquid being water. In certain embodiments, water is present in the liquid in an amount of greater than or equal to 90 wt %, greater than or equal to 91 wt %, greater than or equal to 92 wt %, greater than or equal to 93 wt %, greater than or equal to 94 wt %, greater than or equal to 95 wt %, greater than or equal to 96 wt %, greater than or equal to 97 wt %, greater than or equal to 98 wt %, greater than or equal to 99 wt %, greater than or equal to 99.5 wt %, or greater than or equal to 99.9 wt % versus the total liquid weight. In some embodiments, the liquid comprises less than or equal to 99.99 wt %, less than or equal to 99.9 wt %, less than or equal to 99.5 wt %, less than or equal to 99 wt %, less than or equal to 98 wt %, less than or equal to 97 wt %, less than or equal to 96 wt %, less than or equal to 95 wt %, less than or equal to 94 wt %, less than or equal to 93 wt %, less than or equal to 92 wt %, or less than or equal to 91 wt % water versus the total liquid weight. Combinations of the above-referenced ranges are also possible (e.g., greater than or equal to 90 wt % and less than or equal to 99.99 wt %, greater than or equal to 95 wt % and less than or equal to 99.99 wt %, greater than or equal to 98 wt % and less than or equal to 99.99 wt %). Other ranges are also possible.

In certain embodiments, water is present in the liquid in an amount of greater than or equal to 90 vol %, greater than or equal to 91 vol %, greater than or equal to 92 vol %, greater than or equal to 93 vol %, greater than or equal to 94 vol %, greater than or equal to 95 vol %, greater than or equal to 96 vol %, greater than or equal to 97 vol %, greater than or equal to 98 vol %, greater than or equal to 99 vol %, greater than or equal to 99.5 vol %, or greater than or equal to 99.9 vol % versus the total volume of the liquid. In some embodiments, the liquid comprises less than or equal to 99.99 vol %, less than or equal to 99.9 vol %, less than or equal to 99.5 vol %, less than or equal to 99 vol %, less than or equal to 98 vol %, less than or equal to 97 vol %, less than or equal to 96 vol %, less than or equal to 95 vol %, less than or equal to 94 vol %, less than or equal to 93 vol %, less than or equal to 92 vol %, or less than or equal to 91 vol % versus the total volume of the liquid. Combinations of the above-referenced ranges are also possible (e.g., greater than or equal to 90 vol % and less than or equal to 99.99 vol %, greater than or equal to 95 vol % and less than or equal to 99.99 vol %, greater than or equal to 98 vol % and less than or equal to 99.99 vol %). Other ranges are also possible.

In some embodiments, the composition may be used to improve the health of a subject. For example, the composition may reduce oxidative stress and/or reduce muscle fatigue (e.g., after exercise and/or athletic activity). In certain embodiments, the composition may improve a subject's overall well-being including, for example, a feeling of increased energy levels, hastened recovery after exercise, improved memory, increased strength, and/or reduced tiredness. In some cases, the composition may be particularly delectable to the subject. In addition, in some cases, the composition may be used to treat a disease, disorder, or other clinically recognized condition, or for prophylactic purposes, or for providing physiological benefits, and has a clinically significant effect on the body of the subject to treat and/or prevent the disease, disorder, or condition, and/or has a clinically significant effect on the subject's physiology. In other embodiments, the composition may provide performance enhancement to a subject while, for example, exercising and/or performing athletic activities. In some cases, the aqueous solution may be administered to a subject (e.g., administered to a subject to treat a disease, disorder, or other clinically recognized condition, or for prophylactic purposes). In certain embodiments, the article may be administered orally, intravenously, rectally, nasally (e.g., via a nasal spray, via a nasal dropper), or uretherally (e.g., via a catheter). In addition, in some embodiments, the solution may be self-administered by the subject. In one set of embodiments, a composition as described herein is used to treat a subject, e.g., a human subject. To “treat” a condition means to reduce or eliminate a sign or symptom of the condition, to stabilize the condition, and/or to reduce or slow further progression of the condition. In some cases, the subject may be one that has or is at risk for a disorder or condition. The condition may be, for example, a disease such as discussed herein.

In certain embodiments, the aqueous solution may be orally administered to a subject, (e.g., ingested or drunk by a subject, encapsulated in a pill (e.g., the aqueous solution is contained in a capsule such as a gel-capsule)). In some such embodiments, the aqueous solution may comprise hydrogen gas in an amount of greater than or equal to greater than or equal to 0.1 ppm and less than or equal to 5 ppm and xenon gas (and/or other noble gases) in an amount of greater than or equal to 1 ppm and less than or equal to 20 ppm.

In some embodiments, the aqueous solution may be administered intravenously. In some such embodiments, the aqueous solution comprises hydrogen gas in an amount of greater than or equal to greater than or equal to 0.1 ppm and less than or equal to 5 ppm and xenon gas (and/or other noble gases) in an amount of greater than or equal to 1 ppm and less than or equal to 20 ppm.

In one set of embodiments, the composition, prior to administration to a subject, may be contained within a sealed container, e.g., as described herein. For example, the aqueous solution may be contained within a can, bottle, jar, pouch, box, bag, or capsule. In some cases, the container may be unsealed just before administration to a subject. For example, the container may be unsealed and then administered to a subject (including self-administration) within 1 hour of unsealing the container, or within 45 minutes, within 30 minutes, within 20 minutes, within 15 minutes, within 10 minutes, within 5 minutes, within 4 minutes, within 3 minutes, within 2 minutes, or within 1 minute of unsealing.

In some cases, xenon and other and other noble gases may exhibit synergistic effects with hydrogen (H₂), e.g., for certain applications such as for the treatment of animal and human diseases, for improvement in athletic performance, for the enhancement of the overall health of a subject, or the like. Without wishing to be bound by any theory, while xenon and other noble gases may not be directly involved in any chemical reactions, such gases may participate in physical interactions within a subject (for example, by blocking receptors, creating size exclusion effects, and/or by competing with proteins), thereby resulting in various biological effects. This may be useful, for example, for the treatment of animal and human diseases, for improvement in athletic performance, for the enhancement of the overall health of a subject, or the like.

For example, in one set of embodiments, xenon may be used to induce cardioprotection and/or neuroprotection through a variety of mechanisms. Without wishing to be bound by any theory, it is believed that xenon may affect Ca²⁺, K⁺, KATP/HIF, and/or NMDA antagonism; xenon may also activate PKC-epsilon, p38-MAPK, ATP-sensitive potassium channel, and/or hypoxia inducible factor 1 alpha (HIF1a), thereby allowing cardioprotective and/or neuroprotective effects to occur. In addition, there may be some synergistic effects with hydrogen. Hydrogen may act within the body as an antioxidant, which may treat damage, e.g., caused by oxidative or other stresses, e.g., during ischemia.

Accordingly, in certain cases, a composition as described herein can be used to treat conditions such as ischemia, e.g., partial ischemia or restriction in blood supply to tissues. For instance, a composition may be administered to a subject to protect neural and/or cardiac function. This may be administered on a regular basis, e.g., as discussed herein, and/or in combination with events such as anesthesia, hypoxia, hypothermia, ischemia, oxygen deprivation, oxygen glucose deprivation, exercise, or other similar conditions in which blood and/or oxygen may be lowered or otherwise altered in a subject, permanently or temporarily. Thus, in various embodiments, a subject having or at risk to conditions such as these may be administered (including self-administration) a composition as discussed herein.

In addition, in some cases, hydrogen may be used to treat oxidative stress diseases and conditions such as smoking, exposure to ultraviolet rays, air pollution, aging, physical or psychological stress, or the aging process, e.g., due to its antioxidant properties. Xenon (and/or other noble gases) may facilitate treatment, for example, by increasing red blood cell levels, e.g., as discussed herein.

Accordingly, in one set of embodiments, a composition as described herein is used to treat muscular, neurodegenerative, and/or neuromuscular diseases or other conditions. Thus, in one set of embodiments, the administration of a composition as discussed herein, e.g., comprising hydrogen and/or a noble gas (e.g., xenon) may be administered to a subject. The subject may be one that is suffering from a muscular, neurodegenerative, or neuromuscular disease, and the subject may exhibit clinical improvement after treatment.

In some cases, administration of a composition as discussed herein may act as a preventative of and/or be used to treat a muscular, neurodegenerative, or neuromuscular disease. In another set of embodiments, a composition as described herein is administered to a subject, e.g., one having or at risk of a muscular, neurodegenerative, or neuromuscular disease. In certain embodiments, the disease is a muscular dystrophy or atrophy (e.g., Becker's muscular dystrophy, congenital muscular dystrophy, Duchenne muscular dystrophy, distal muscular dystrophy, Emery-Dreifuss muscular dystrophy, facioscapulohumeral muscular dystrophy, limb-girdle muscular dystrophy, myotonic muscular dystrophy, oculopharyngeal muscular dystrophy, spinal muscular atrophy, Brown-Vialetto-Van Laere syndrome, Fazio-Londe syndrome). In some embodiments, the disease is a muscular atrophy (e.g., muscle atrophy associated with a cancer, muscle atrophy associated with AIDS, muscle atrophy associated with congestive heart failure, muscle atrophy associated with chronic obstructive pulmonary disease, muscle atrophy associated with renal failure, muscle atrophy associated with severe burns, and muscle atrophy associated with long bed rest). In certain embodiments, the disease is amyotrophic lateral sclerosis. In some embodiments, the disease is Charcot-Marie-Tooth disease, Dejerine-Sottas disease, or Kennedy's disease. In some cases, the disease is Parkinson's disease, Alzheimer's disease, or Huntington's disease. In some embodiments, the subject may be suffering or at risk of neuropathic pain.

In some cases, the subject may be one that already has a muscular, neurodegenerative, or neuromuscular disease. However, in other cases, the subject may not necessarily have a muscular, neurodegenerative, or neuromuscular disease, but may be one that is at risk of developing such a disease. In some embodiments, the subject suffers from a muscular dystrophy. In some embodiments, the subject suffers from muscular atrophy. In some embodiments, the subject is in need of muscle growth or repair. In some embodiments, the subject is in need of enhanced muscle performance.

In some embodiments, compositions described herein may be used for stimulation of vasodilation prior to physical exercise. The composition may be used, for example, prior to a workout at the gym, or before a training or game of any sports. In some embodiments, a composition described herein is administered to a subject prior to physical exercise. In certain embodiments, compositions described herein are administered to provide enhanced muscle performance.

The subject may be a healthy subject, or one who has or is at risk for a muscular or neuromuscular disease, e.g., as discussed herein. In some cases, the subject may be an older subject, e.g., having or at risk of muscle weakening due to age. For example, the subject may be one that is at least 40 years old, at least 50 years old, at least 60 years old, or at least 70 years old.

In another set of embodiments, the present invention is generally directed to preventing and/or reversing cardiovascular disease, such as atherosclerosis, hypertension, high blood pressure, sickle-cell anemia, neointimal hyperplasia, peripheral artery disease, high-density lipoprotein deficiency, etc., e.g., using compositions and methods as discussed herein.

In some embodiments, the subject may be one that exhibits one or more symptoms of atherosclerosis. For example, the subject may have a history or a family history of atherosclerosis, or the subject may exhibit symptoms such as elevated blood pressure (i.e., hypertension), chest pain (angina), sudden numbness or weakness in the arms or legs, difficulty speaking or slurred speech, drooping muscles in the face, leg pain when walking, and/or claudication.

Additionally, in some embodiments, the composition may be applied in conjunction with other types of treatments to a subject, e.g., to treat or prevent arteriosclerosis, hypertension, sickle-cell anemia, etc. Non-limiting examples of such treatments include any one or more of those discussed herein. These may be occur, e.g., simultaneously or sequentially, in various embodiments.

Examples of other treatments of cardiovascular diseases include, but are not limited to, nitrates (e.g., nitroglycerine, isosorbide, etc.), beta blockers (e.g., atenolol, metoprolol, nadolol, oxprenolol, pindolol, propranolol, timolol, etc.), alpha blockers (e.g., doxazosin, phentolamine, indoramin, phenoxybenzamine, prazosin, terazosin, tolazoline, etc.), calcium channel blockers (e.g., amlodipine, aranidipine, azelnidipine, barnidipine, benidipine, cilnidipine, clevidipine, isradipine, efonidipine, felodipine, lacidipine, lercanidipine, manidipine, nicardipine, nifedipine, nilvadipine, nimodipine, nisoldipine, nitrendipine, pranidipine, diltiazem, mibefradil, bepridil, fluspirilene, fendiline, etc.), or the like.

Additional examples of treatments include, but are not limited to, loop diuretics (e.g., bumetanide, ethacrynic acid, furosemide, torsemide, etc.), thiazide diuretics (e.g., epitizide, hydrochlorothiazide, chlorothiazide, bendroflumethiazide, etc.), thiazide-like diuretics (e.g., indapamide, chlorthalidone, metolazone, etc.), potassium-sparing diuretics (e.g., amiloride, triamterene, spironolactone, etc.), beta blockers (e.g., atenolol, metoprolol, nadolol, oxprenolol, pindolol, propranolol, timolol, etc.), alpha blockers (e.g., doxazosin, phentolamine, indoramin, phenoxybenzamine, prazosin, terazosin, tolazoline, etc.), mixed alpha and beta blockers (e.g., bucindolol, carvedilol, labetalol, etc.), dihydropyridines (e.g., amlodipine, felodipine, isradipine, lercanidipine, nicardipine, nifedipine, nimodipine, nitrendipine, etc.), non-dihydropyridines (e.g., diltiazem, verapamil, etc.), ACE inhibitors (e.g., captopril, enalapril, fosinopril, lisinopril, perindopril, quinapril, ramipril, trandolapril, benazepril, etc.), angiotensin II receptor antagonists (e.g., candesartan, eprosartan, irbesartan, losartan, olmesartan, telmisartan, valsartan, etc.), aldosterone receptor antagonists (e.g., eplerenone, spironolactone, etc.), vasodilators (e.g., sodium nitroprusside, hydralazine, etc.), alpha-2 agonists (e.g., clonidine, guanabenz, methyldopa, moxonidine, etc.), adrenergic neuron blockers (e.g., guanethidine, reserpine, etc.), or the like.

In yet another set of embodiments, a composition as discussed herein may be administered to a subject, for example, to improve athletic performance. For example, strenuous exercise may cause oxidative stresses, e.g., due to muscle fatigue. Compositions as discussed herein may be useful to reduce oxidative damage during exercise. In addition, in some cases, xenon may be used to increase production of erythropoietin. This may be useful, for example, to increase red blood cells, e.g., to treat anemic subjects, or improve athletic performance. Without wishing to be bound by any theory, it is believed that xenon may enhance production of HIF1a, which is a transcription factor able to respond to hypoxic conditions. Thus, in some cases, a composition as described herein may be used to increase a subject's physical energy levels, e.g., for improvement in athletic performance. Additionally, in some embodiments, a composition as described herein can be used to treat anemia or other conditions in a subject.

In addition, in low doses, xenon may cause certain analgesic effects, which may facilitate improved athletic performance in some cases (e.g., due to lower or reduced pain). For example, xenon may inhibit nicotinic acetylcholine alpha-4-beta-2 (α₄β₂) receptors, plasma membrane Ca²⁺ ATPase, and/or the serotonin 5-HT3 receptor. Xenon may also be an antagonist of high-affinity glycine-site NMDA, or it may activate the two-pore domain potassium channel TREK-1.

Additionally, in one set of embodiments, a composition as discussed herein may be applied to a subject that is about to be anaesthetized (partially or completely), for example, by applying a suitable anesthetic to the subject (e.g., general or regional anesthetics). The composition may provide, for example, various neuroprotective effects, or other effects such as those described herein. The composition and the anesthetic may be administered to the subject in any suitable order, e.g., simultaneously or sequentially (in any order). In some cases, the subject is conscious while the composition is administered (for example, the subject may drink the composition). In some cases, the anesthetic and the composition are administered proximate in time. For instance, the anesthetic and the composition can be administered such that the subject will be anesthetized while subject to at least some of the effects of the composition (e.g., comprising hydrogen and/or a noble gas such as xenon gas). In some cases, the anesthetic and the composition may be administered within 60 minutes, 45 minutes, 30 minutes, 20 minutes, 15 minutes, 10 minutes, or 5 minutes of each other. Examples of anesthetics that may be administered include, but are not limited to, propofol, sodium thiopental, etomidate, ketamine, sevoflurane, lidocaine, fentanyl, nitrous oxide, isoflurane, or desflurane. In addition, in some embodiments, xenon itself may be provided as an anesthetic.

Another set of embodiments of the present invention permit treatment or prevention of various symptoms associated with metabolic conditions or disorders, e.g., diabetes or excessive weight gain. In some embodiments, metabolic conditions generally relate to conditions or disorders that that interfere with the body's metabolism. In some embodiments, metabolic conditions are inherited. Some metabolic disorders can be diagnosed by routine screening tests done at birth. Others are identified only after a child or adult shows symptoms of a disorder. Examples of metabolic conditions, disorders, or diseases contemplated by the present invention include, but are not limited to, obesity, diabetes, metabolic syndrome, impaired glucose tolerance (IGT), hyperglycemia, insulin resistance, dyslipidemia, or the like.

In certain embodiments, compositions provided herein are useful in treating metabolic syndrome, which in some aspects, is defined by The National Cholesterol Education Program criteria, which is defined as the presence of three or more of the following risk factors in the same individual: abdominal obesity or waist circumference greater than 102 cm (40 in) (men) or greater than 88 cm (35 in) (women), serum triglycerides greater or equal to 150 mg/dl, HDL cholesterol less than 40 mg/dl (men) or less than 50 mg/dl (women), systolic blood pressure greater than or equal to 130 mm Hg, diastolic blood pressure greater than or equal to 85 mm Hg, fasting blood glucose greater than or equal to 110 mg/dl. Thus, in some embodiments, the effectiveness of certain compositions, as described herein, is monitored using the above criteria, e.g., by observing a decrease in one or more risk factors over time.

In another set of embodiments, a composition as discussed herein may be administered to a subject to improve metabolism within the subject. In some cases, for instance, there may be antiaging or other positive effects, such as increased muscle growth, bone density, cartilage strength, tendon strength, or the like. In addition, there may be improved metabolism of various systems, such as the kidney, pancreas, gonads, or the like.

One set of embodiments is generally directed to treatment of various inflammatory diseases, such as inflammatory dermatoses, arthritis, osteoarthritis, septic shock, rheumatoid arthritis, or other autoimmune diseases. Without wishing to be bound by any theory, it is believed that xenon may increase removal of selectin PSGL-1 and L-selectin, and thus, xenon may have anti-inflammatory properties, as these selectins are part of the inflammatory response. In addition, xenon may limit injuries such as myocardial, brain, lung and/or kidney injury through inhibition of the N-methyl-d-aspartate (NMDA) receptor, for example, caused by hypothermia, hypoxia, ischemia, oxygen deprivation, oxygen glucose deprivation, or the like, e.g., by limiting glutamate excitotoxicity. In addition, without wishing to be bound by any theory, hydrogen may also exhibit anti-inflammatory effects, for example, by inducing inflammatory cytokines and/or decreasing the expressions of pro-inflammatory factors such as TNF-alpha, IL-6, IL-1-beta, CCL2, IL-10, TNF-gamma, IL-12, ICAM-1, HMGB-1, NF-kB, PGE2, etc. Thus, it is believed that the combination of xenon and hydrogen may have relatively large anti-inflammatory effects, which may be useful for treating various inflammatory diseases such as those discussed herein.

In certain embodiments of the invention, the administration of various compositions of the invention may be designed so as to result in sequential exposures to the composition over a certain time period, for example, hours, days, weeks, months, or years. This may be accomplished, for example, by repeated administrations of a composition of the invention by one or more of the methods described herein. In some cases, compositions may be applied to the subject on a relatively regular or periodic basis; e.g., a subject may drink a container each day, or a two, three, four, or more containers a day, or a container every other day, every third day, every fourth day, etc. Somewhat more irregular schedules are also possible (e.g., a regular number of containers per week or per month, etc.).

Thus, the compositions of the present invention may be administered in multiple doses over extended period of time. For any composition described herein the therapeutically effective amount can be initially determined from animal models. The applied dose can be adjusted based on the relative bioavailability and potency of the administered composition. Adjusting the dose to achieve maximal efficacy based on the methods described above and other methods as are well-known in the art is well within the capabilities of the ordinarily skilled artisan.

When administered to a subject, effective amounts will depend on the particular condition being treated and the desired outcome. A therapeutically effective dose may be determined by those of ordinary skill in the art, for instance, employing factors such as those described herein and using no more than routine experimentation.

In administering the compositions of the invention to a subject, dosing amounts, dosing schedules, routes of administration, and the like may be selected so as to affect known activities of these compositions. Dosages may be estimated based on the results of experimental models, optionally in combination with the results of assays of compositions of the present invention. Dosage may be adjusted appropriately to achieve desired drug levels, local or systemic, depending upon the mode of administration. The doses may be given in one or several administrations per day. Multiple doses per day are also contemplated in some cases to achieve appropriate systemic levels of the composition within the subject or within the active site of the subject.

The dosage may be given in some cases at the maximum amount while avoiding or minimizing any potentially detrimental side effects within the subject. The dosage of the composition that is actually administered is dependent upon factors such as the final concentration desired at the active site, the method of administration to the subject, the efficacy of the composition, the longevity of the composition within the subject, the timing of administration, the effect of concurrent treatments (e.g., as in a cocktail), etc. The dose delivered may also depend on conditions associated with the subject, and can vary from subject to subject in some cases. For example, the age, sex, weight, size, environment, physical conditions, or current state of health of the subject may also influence the dose required and/or the concentration of the composition. Variations in dosing may occur between different individuals or even within the same individual on different days. It may be preferred that a maximum dose be used, that is, the highest safe dose according to sound medical judgment. Preferably, the dosage form is such that it does not substantially deleteriously affect the subject.

Administration of the composition can be alone, or in combination with other therapeutic agents and/or compositions. In certain embodiments of the invention, a composition can be combined with a suitable pharmaceutically acceptable carrier, for example, within a suitable liquid. In general, pharmaceutically acceptable carriers suitable for use in the invention are well-known to those of ordinary skill in the art. As used herein, a “pharmaceutically acceptable carrier” refers to a non-toxic material that does not significantly interfere with the effectiveness of the biological activity of the active composition(s) to be administered, but is used as a formulation ingredient, for example, to stabilize or protect the active composition(s) within the composition before use. The carrier may include one or more compatible solid or liquid fillers, diluents or encapsulating substances which are suitable for administration to a human or other vertebrate animal. The term “carrier” denotes an organic or inorganic ingredient, which may be natural or synthetic, with which one or more compositions of the invention are combined to facilitate the application of the composition. The carrier may be co-mingled or otherwise mixed with one or more compositions of the present invention, and with each other, in a manner such that there is no interaction which would substantially impair the desired pharmaceutical efficacy. Those skilled in the art will know of suitable carriers, such as saline, or will be able to ascertain such, using only routine experimentation.

The formulations of the invention are administered in pharmaceutically acceptable solutions, which may routinely contain pharmaceutically acceptable concentrations of salt, buffering agents, preservatives, compatible carriers, adjuvants, emulsifiers, diluents, excipients, chelating agents, fillers, drying agents, antioxidants, antimicrobials, preservatives, binding agents, bulking agents, silicas, solubilizers, stabilizers and optionally other therapeutic ingredients, that may be used with the active composition. For example, if the formulation is a liquid, the carrier may be a solvent, partial solvent, or non-solvent, and may be aqueous or organically based. Examples of suitable formulation ingredients include diluents such as calcium carbonate, sodium carbonate, lactose, kaolin, calcium phosphate, or sodium phosphate; granulating and disintegrating agents such as corn starch or algenic acid; binding agents such as starch, gelatin or acacia; lubricating agents such as magnesium stearate, stearic acid, or talc; time-delay materials such as glycerol monostearate or glycerol distearate; suspending agents such as sodium carboxymethylcellulose, methylcellulose, hydroxypropylmethylcellulose, sodium alginate, polyvinylpyrrolidone; dispersing or wetting agents such as lecithin or other naturally-occurring phosphatides; thickening agents such as cetyl alcohol or beeswax; buffering agents such as acetic acid and salts thereof, citric acid and salts thereof, boric acid and salts thereof, or phosphoric acid and salts thereof; or preservatives such as benzalkonium chloride, chlorobutanol, parabens, or thimerosal. Suitable carrier concentrations can be determined by those of ordinary skill in the art, using no more than routine experimentation. The compositions of the invention may be formulated into preparations in solid, semi-solid, liquid or gaseous forms such as tablets, capsules, elixirs, powders, granules, ointments, solutions, depositories, inhalants or injectables. Those of ordinary skill in the art will know of other suitable formulation ingredients, or will be able to ascertain such, using only routine experimentation.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

Preparations include sterile aqueous or nonaqueous solutions, suspensions and emulsions, which can be isotonic with the blood of the subject in certain embodiments. Examples of nonaqueous solvents are polypropylene glycol, polyethylene glycol, vegetable oil such as olive oil, sesame oil, coconut oil, arachis oil, peanut oil, mineral oil, injectable organic esters such as ethyl oleate, or fixed oils including synthetic mono or di-glycerides. Aqueous carriers include water, alcoholic/aqueous solutions, emulsions or suspensions, including saline and buffered media. Parenteral vehicles include sodium chloride solution, 1,3-butandiol, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. Preservatives and other additives may also be present such as, for example, antimicrobials, antioxidants, chelating agents and inert gases and the like. Those of skill in the art can readily determine the various parameters for preparing and formulating the compositions of the invention without resort to undue experimentation.

The present invention also provides any of the above-mentioned compositions in kits, optionally including instructions for use of the composition for the treatment of a condition discussed herein. That is, the kit can include a description of use of the composition for participation in any biological or chemical mechanism disclosed herein associated with a condition discussed herein. The kit can include a description of use of the compositions as discussed herein. The kit also can include instructions for use of a combination of two or more compositions of the invention, or instruction for use of a combination of a composition of the invention and one or more other compositions. Instructions also may be provided for administering the composition by any suitable technique as previously described, for example, orally, intravenously, pump or implantable delivery device, or via another known route of drug delivery.

The kits described herein may also contain one or more containers, which may contain the composition and other ingredients as previously described. The kits also may contain instructions for mixing, diluting, and/or administrating the compositions of the invention in some cases. The kits also can include other containers with one or more solvents, surfactants, preservative and/or diluents (e.g., normal saline (0.9% NaCl), or 5% dextrose) as well as containers for mixing, diluting or administering the components in a sample or to a subject in need of such treatment.

The compositions of the kit may be provided as any suitable form, for example, as liquid solutions. In embodiments where liquid forms of the composition are used, the liquid form may be concentrated or ready to use. The solvent will depend on the composition and the mode of use or administration. Suitable solvents for drug compositions are well known, for example as previously described, and are available in the literature. The solvent will depend on the composition and the mode of use or administration.

In certain embodiments, the articles, compositions, and aqueous solutions described herein are substantially non-toxic. The term “non-toxic” refers to a substance that does not comprise a toxic compound. The term “toxic” refers to a substance showing detrimental, deleterious, harmful, or otherwise negative effects on a subject, tissue, or cell when or after administering the substance to the subject or contacting the tissue or cell with the substance, compared to the subject, tissue, or cell prior to administering the substance to the subject or contacting the tissue or cell with the substance. In certain embodiments, the effect is death or destruction of the subject, tissue, or cell. In certain embodiments, the effect is a detrimental effect on the metabolism of the subject, tissue, or cell. In certain embodiments, a toxic substance is a substance that has a median lethal dose (LD50) of not more than 500 milligrams per kilogram of body weight when administered orally to an albino rat weighing between 200 and 300 grams, inclusive. In certain embodiments, a toxic substance is a substance that has an LD50 of not more than 1,000 milligrams per kilogram of body weight when administered by continuous contact for 24 hours (or less if death occurs within 24 hours) with the bare skin of an albino rabbit weighing between two and three kilograms, inclusive. In certain embodiments, a toxic substance is a substance that has an LC50 in air of not more than 2,000 parts per million by volume of gas or vapor, or not more than 20 milligrams per liter of mist, fume, or dust, when administered by continuous inhalation for one hour (or less if death occurs within one hour) to an albino rat weighing between 200 and 300 grams, inclusive.

A “subject” refers to any animal such as a mammal (e.g., a human). Non-limiting examples of subjects include a human, a non-human primate, a cow, a horse, a pig, a sheep, a goat, a dog, a cat or a rodent such as a mouse, a rat, a hamster, a bird, a fish, or a guinea pig. Generally, the invention is directed toward use with humans. In some embodiments, a subject may demonstrate health benefits, e.g., upon administration of the liquid. As used herein, a “fluid” is given its ordinary meaning, i.e., a liquid or a gas. A fluid cannot maintain a defined shape and will flow during an observable time frame to fill the container in which it is put. Thus, the fluid may have any suitable viscosity that permits flow. If two or more fluids are present, each fluid may be independently selected among essentially any fluids (liquids, gases, and the like) by those of ordinary skill in the art.

U.S. Patent Application Ser. No. 62/510,102, filed on May 23, 2017, entitled “Water and Other Liquids Containing Hydrogen and/or Noble Gases,” by Perricone, is incorporated herein by reference in its entirety. In addition, U.S. patent application Ser. No. 15/834,262, filed on Dec. 7, 2017, entitled “Water and Other Liquids Containing Hydrogen and/or Noble Gases,” by Perricone, and U.S. Provisional Patent Application Ser. No. 62/510,114, filed May 23, 2017, entitled “Systems and Methods for Treatments Using Hydrogen and/or Noble Gases,” by Perricone, are each incorporated herein by reference in its entirety.

### EXAMPLES

The following examples illustrate embodiments of certain aspects of the invention. It should be understood that the methods and/or materials described herein may be modified and/or scaled, as known to those of ordinary skill in the art.

### Prophetic Example 1

A container contains a liquid comprising water, hydrogen gas, and xenon gas. The hydrogen gas is present in an amount of greater than or equal to 1.5 ppm and less than or equal to 2 ppm. The xenon gas is present in an amount of greater than 10 ppm and less than 15 ppm. The liquid is administered to a subject (e.g., orally, intravenously) and the subject receives the benefits of the liquid, or hydration from the liquid itself.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed:

1-99. (canceled)

100. A method, comprising:
administering, to a subject in need of a neuroprotectant, an aqueous solution comprising dissolved hydrogen gas and dissolved xenon gas.

101. The method of claim 100, wherein administering the aqueous solution to the subject comprises oral administration.

102. The method of claim 100, wherein the subject self-administers the aqueous solution.

103. The method of claim 100, wherein the aqueous solution is at least 90 wt % water.

104. The method of claim 100, wherein the aqueous solution consists essentially of water.

105. The method of claim 100, wherein the dissolved xenon gas is at a concentration of at least 1 ppm.

106. The method of claim 100, wherein the dissolved hydrogen gas is at a concentration of at least 1 ppm.

107. The method of claim 100, wherein the dissolved hydrogen gas is at a concentration of at least 3 ppm.

108. The method of claim 100, wherein the aqueous solution, prior to administration, is contained within a sealed container.

109. The method of claim 108, wherein the sealed container has a pressure at least 1 psi greater than atmospheric pressure.

110. A method, comprising:
administering, to a subject having or at risk of neurodegeneration, an aqueous solution comprising dissolved hydrogen gas and dissolved xenon gas.

111. The method of claim 110, wherein administering the aqueous solution to the subject comprises oral administration.

112. The method of claim 110, wherein the subject self-administers the aqueous solution.

113. The method of claim 110, wherein the aqueous solution is at least 90 wt % water.

114. The method of claim 110, wherein the aqueous solution consists essentially of water.

115. The method of claim 110, wherein the dissolved xenon gas is at a concentration of at least 1 ppm.

116. The method of claim 110, wherein the dissolved hydrogen gas is at a concentration of at least 1 ppm.

117. The method of claim 110, wherein the dissolved hydrogen gas is at a concentration of at least 3 ppm.

118. The method of claim 110, wherein the aqueous solution, prior to administration, is contained within a sealed container.

119. The method of claim 118, wherein the sealed container has a pressure at least 1 psi greater than atmospheric pressure.
